(19) United States
(12) Patent Application Publication
(10) Pub. No.: US 2022/0040364 A1
(43) Pub. Date: Feb. 10, 2022

(54) Systems for Avoiding Contamination and Accumulation of Airborne Pathogens

(71) Applicant: Ferdinand Romano, Cocoa, FL (US)

(72) Inventors: Rainer B. Meinke, Melbourne, FL (US); Ferdinand M. Romano, Merritt Island, FL (US)

(21) Appl. No.: 17/495,110

(22) Filed: Oct. 6, 2021

Related U.S. Application Data
(60) Provisional application No. 63/063,235, filed on Aug. 8, 2020.

(51) Int. Cl. A61L 9/20; A61L 9/18

(52) U.S. Cl. CPC A61L 9/20; A61L 2209/12; A61L 9/18

(57) ABSTRACT

In one embodiment, systems and methods for reducing infections from a pathogen by influencing transport of the pathogen. A material is positioned in a location where a pathogen may be transmitted from a person and through a volume of air about the person. The material is provided with a net electric charge sufficient to create a measurable electric field extending from the material. To the extent the pathogen becomes positioned along a portion of the material, a treatment is applied about the material portion to disable the infectious nature of the pathogen.

### RELATED APPLICATION

This application claims priority to U.S. Provisional Patent Application Ser. No. 63/063,235 titled “Integrated System for Avoiding Contamination and Accumulation of Airborne Pathogens in Closed Environments”, filed Aug. 8, 2020.

### FIELD OF THE INVENTION

The invention relates to systems and processes for avoiding infectious diseases and, more specifically, discloses systems and methods for sanitizing viral and bacterial pathogens.

### BACKGROUND

It is widely believed that the COVID-19 like influenza-A viruses and other viruses spread among humans through cyclic exposures to airborne respiratory droplets expelled from individuals during coughing, sneezing and even speaking. Viruses can also survive on surfaces for many hours and can thereby be otherwise transferred to people who, often unknowingly, eventually transfer the infectious species from their respiratory systems. This infectious spreading can be mitigated by frequent hand washing, avoiding contact between the hands, nose and eyes and use of face masks. The mode of virus transmission occurring through airborne droplets may be the most likely transport means responsible for infections by the so-called “super spreaders”, i.e., individuals who infect a large number of other people. Given recent evidence, that persons who have received COVID-19 vaccinations may play a significant role in spreading the more infectious variants of SARS-COV-2, there also appears to be an unmet need to more effectively control the compounded rates at which airborne viruses appear to spread. The recommendation of the World Health Organization (WHO) for keeping a distance of about 2 m between people is based in part on experiments performed in the 1930's by William Wells, a Harvard researcher who studied tuberculosis. [See, Katherine Ellen Foley. “Where does the six-foot guideline for social distancing come from?”, Quartz Media Inc, Apr. 15,2020, url: https://qz.com/1831100/where-does-the-six-feet-social-distancing-guideline-come-from/; See, W. F. Wells, “ON AIR-BORNE INFECTION: STUDY II. DROPLETS AND DROPLET NUCLEI”, American Journal of Epidemiology, Volume 20, Issue 3, November 1934, Pages 611-618, url: https://doi.org/10.1093/oxfordjournals.aje.a118097].

Thus a relevant distinction appears to exist between (i) larger droplets, believed to settle to the ground within 1-2 meters of a source, based on gravity, and (ii) smaller droplet particles with aerodynamic diameters near or below 1 μm that can remain airborne for long periods of time and travel over large distances. It is now well-known that these latter particles, conventionally referred to as aerosols and micro-droplets, having diameters up to a few μm, can contain pathogens, including viruses, and can be catapulted for more than 6 m. Aerosol transmission appears relevant to spread of viral pathogens in closed environments. Susceptibility to infection by aerosol or micro-droplet transmission in closed rooms can be up to 20 times higher [See, Kim Sneppen, Bjarke Frost Nielsen, Robert J. Taylor, Lone Simonsen, “Overdispersion in COVID-19 increases the effectiveness of limiting nonrepetitive contacts for transmission control”, Proceedings of the National Academy of Sciences, Volume 118, Issue 14, April 2021, e2016623118; DOI: 10.1073/pnas.2016623118] than in an open environment.

For influenza, the importance of bio-aerosol transmission was confirmed in controlled experiments in a simulated patient examination room using coughing and breathing mannequins to determine whether coughed influenza was infectious [See, John D. Noti, et al., “Detection of Infectious Influenza Virus in Cough Aerosols Generated in a Simulated Patient Examination Room, Clinical Infectious Diseases”, Volume 54, Issue 11, 1 Jun. 2012, Pages 1569-1577, url: https://doi.org/10.1093/cid/cis237].

Aerosol particle counters were used to measure aerosol concentrations at locations throughout the test chamber. Dispersion of aerosol particles, e.g., water droplets, with diameters from 0.3 micron to 7.5 microns was evaluated along with the influence of breathing rate, room ventilation, and the locations of the coughing and breathing simulators. Results showed that cough-generated aerosol particles spread rapidly throughout the room and, within 5 minutes, a person anywhere in the room is exposed to potentially hazardous aerosols.

A significant proportion of individuals infected with COVID-19 may not have symptoms, raising the likelihood of disease transmission, not only to inadequately protected health care workers but, also, among general populations in offices, restaurants, churches, schools and other situations where groups of people gather in closed environments for extended periods of time.

The spread of COVID-19 has been found more pervasive than influenza, in part because super spreaders appear more responsible for the spread of infection among affected population groups for COVID-19 than for influenza. The extent of the pervasiveness may be quantitatively described by the dispersion factor (k), which describes how much or quickly a disease manifests among a population group. The lower the k-factor, the more the transmission comes from a smaller number of people. While in the flu pandemic of 1918, the k-value was about one, in a recent preprint Adam Kucharski of of the London School of Hygiene & Tropical Medicine estimated that the k-value for COVID-19 is as low as 0.1 [See, Endo A, Centre for the Mathematical Modelling of Infectious Diseases COVID-19 Working Group, Abbott S et al., “Estimating the overdispersion in COVID-19 transmission using outbreak sizes outside China” [version 3; peer review: 2 approved]. Wellcome Open Research, 5:67, April 2020, url: https://doi.org/10.12688/wellcomeopenres.15842.3]. His finding is summarized in the journal stating: “Probably about 10% of cases lead to 80% of the spread”.

### SUMMARY OF THE INVENTION

The system now disclosed significantly reduces the probability of viral transmission through aerosols in closed rooms. To reduce the probability of infection in various environments, where groups of people gather for extended periods of time, pathogen contamination and accumulation must be avoided, and passage of pathogen-containing aerosols from person to person must be mitigated or totally blocked. With the disclosed invention, this is effectively achieved by introducing localized air draft systems that are close to people and directing these air drafts to modular system units that capture and effectively neutralize potential pathogens. The disclosed system can be introduced in existing environments like schools, offices, dental offices, restaurants, etc., does not interfere with existing air conditioning installations and will significantly reduce the probability of person-to-person transmission of infectious disease.

In one embodiment a method is provided for reducing the from a pathogen by influencing transport with an electric field effect based on a charge associated with the pathogen or responsive to a electric charge associated with a particle comprising the pathogen. The effect may be based on net charge or based on charge polarization between or among atoms or molecules associated with the pathogen or the particle. The method includes positioning a material in a location where the pathogen may be transmitted from a person and through a volume of air about the person. The material is provided with a net electric charge sufficient to create a measurable electric field extending from the material. To the extent the pathogen becomes positioned along a portion of the material, a treatment is applied about the material portion to disable the infectious nature of the pathogen. The treatment may be provided to the material portion by transporting the material portion to a region which isolates the effects of the treatment from one or more persons present about the volume of air. The pathogen may be part of an aerosol particle.

An embodiment of a treatment system for rendering a pathogen non-infectious within an open area, there is provided a sheet positionable in the open area and through which an air flow can pass. The system includes a ventilation device positionable to impart an air flow between a person present in the open area and the sheet to direct movement of air emitted by the person toward the sheet. A mechanism or characteristic associated with the sheet is of sufficient strength to cause attachment to the sheet of a particle in the air flow which includes the pathogen, The system further includes a radiant source positioned to provide a sufficient dose of radiant flux directed to the sheet such that, when the pathogen is attached to the sheet, the radiation dose renders the pathogen non-infectious. The open area may be indoors, e.g., a large room, or outdoors. The sheet may be rotatable to sequentially move portions of the sheet past the radiant source. The sheet may comprise any of a porous fabric, a woven fabric, and a screen, mesh or fabric comprising copper.

In another embodiment, a treatment process for rendering a pathogen non-infectious includes, in a predefined area, actively redirecting an air flow emitted by a person toward a sheet exposed to an air current in the predefined area and through which a portion of the air flow and air current can pass. When a pathogen is present in the air flow it is redirected by the air current toward the sheet, attaching the pathogen to the sheet. While the pathogen is on the sheet, a treatment process is applied directly to the pathogen to render the pathogen non-infectious. The attaching may result from a force due to presence of an electric field, surface tension or capillary effects.

A system is provided to reduce infectious contact of an airborne pathogen with a second person, after the pathogen is transmitted from a first person and through a volume of air. The system includes a substrate suitable to receive and retain the pathogen at a position thereon, and a source configured to provide energy to the pathogen's position and render the pathogen noninfectious while the pathogen is on the substrate. In an embodiment of the system, the substrate comprises a sheet of nonporous material which includes multiple positions at which the pathogen may be retained. The sheet may comprise a vinyl nonwoven layer. The substrate may comprise a porous sheet of material through which air can pass, the sheet including multiple positions at which the pathogen may be retained. The sheet may be a fabric or may comprise woven or knitted synthetic fibers. The system may be configured so that when the pathogen is being received at a position on the sheet, air which carries the pathogen to the position passes through the sheet. The sheet may be a woven cloth comprising copper. For example, the sheet may be a woven cloth comprising fiber clad with copper. The sheet may be a HEPA filter or a MERV filter, which captures airborne pathogens. In still other embodiments, the sheet may be a screen or a fabric comprising a series of consecutive, equally spaced-apart threads and the thread pitch, or distance between an adjoining pair of the threads, may range from 0.2 mm to 0.5 mm. The sheet may be a screen having an exterior surface comprising copper. The substrate may comprise a porous sheet having an outer surface comprising copper. The sheet may comprise a copper exterior surface which includes a plurality of apertures to permit flow of air through the sheet so that, when the pathogen is being received at a position on the sheet, a flow of air which carries the pathogen to the position passes through the porous sheet. The sheet may comprise a mesh screen, or a plurality of fibrous threads extending in multiple directions, or interwoven fibers.

The afore described system may include a pair of spaced-apart rollers, where the substrate comprises an insulative layer positioned to extend about the rollers for rotational movement, and the source is positioned to direct radiant flux toward a first of the rollers. In such an embodiment, the insulative layer may be a fabric, a rubber or may comprise a synthetic nylon or polyester fiber. The system may further include a shield positioned adjacent the first roller to limit emanation of the radiant flux in directions away from the first roller to block the radiant flux from contacting persons positioned near the system; or may include a mechanism to accumulate electrical charge on the substrate so that the substrate, when charged, sustains an electric field extending away from a major surface of the substrate to influence movement of the pathogen toward the major surface. The mechanism may include a charge generator coupled to accumulate charge on the substrate where, when charged, the material layer sustains a net charge which generates an electric field extending away from the major surface of the layer.

According to another series of embodiments, a system is provided to reduce contact with an infectious airborne pathogen transmitted from a person. The system may include a filter that traps infectious pathogens, and a source positioned to provide energy to the filter that renders the trapped pathogens noninfectious while the pathogens are trapped in the filter. In one example, the source irradiates the trapped pathogens with UV-C light. The source may be positioned to provide radiant energy within a shielded cavity to irradiate only a portion of the layer positioned within the cavity to render the pathogen noninfectious. The system may further include a pair of spaced apart members, with a first of the members in the form of a shield defining a cavity, or otherwise having a cavity therein, and with the first member having at least one opening suitable to receive the layer into, and convey the layer out of, the first member; with the source positioned to provide radiant energy within the cavity and to a portion of the layer positioned within the cavity to render the pathogen, when placed thereon, noninfectious.

### DESCRIPTION OF THE DRAWINGS

Features, and advantages of the present invention will be better understood when the following detailed description is read with reference to the accompanying drawings, wherein:

FIG. 1A is a schematic diagram of a system comprising modular electrostatic capture units shown installed in a dining room;

FIG. 1B is a plan view (from above) of the room shown in FIG. 1A further illustrating the modular electrostatic capture units installed along a wall, ceiling and floor;

FIG. 2A is a front elevation view of an electrostatic capturer unit according to an embodiment of the invention (Rainer's updated version of left side of original FIG. 2 in provisional);

FIG. 2B is a side elevation view of the electrostatic capturer unit of FIG. 2A;

FIG. 3A is a front elevation view of an electrostatic capturer unit according to another embodiment of the invention;

FIG. 3B is a side elevation view of the electrostatic capturer unit of FIG. 3A;

FIG. 4A is a front elevation view of an electrostatic capturer unit according to still another embodiment of the invention;

FIG. 5 illustrates an electrostatic catcher implementation for people sitting opposite each other with vertical airflow;

FIG. 6 illustrates another electrostatic catcher implementation for people sitting opposite each other with vertical airflow;

FIG. 7 illustrates still an electrostatic catcher implementation for multiple tables with people sitting opposite each other with vertical airflow and separating wall between tables;

FIG. 8 illustrates a booth table with an airflow system integrated therein;

FIG. 9 illustrates a school classroom having desks with integrated airflow systems;

FIG. 10 illustrates a high table for bar with integrated airflow system; and

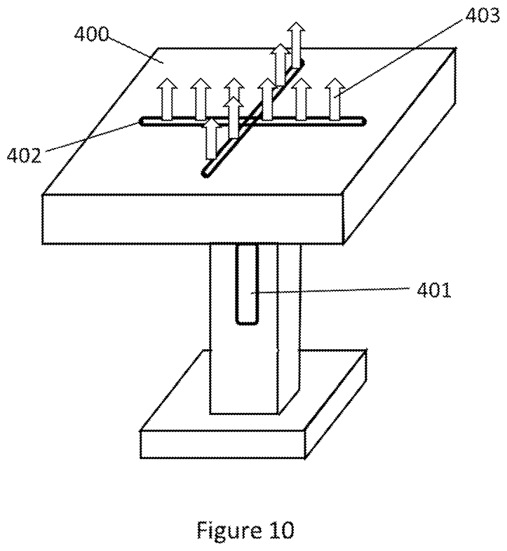

FIG. 11 Illustrates a large room equipped with a central airflow stand and airflow from the walls and multiple electrostatic capture units.

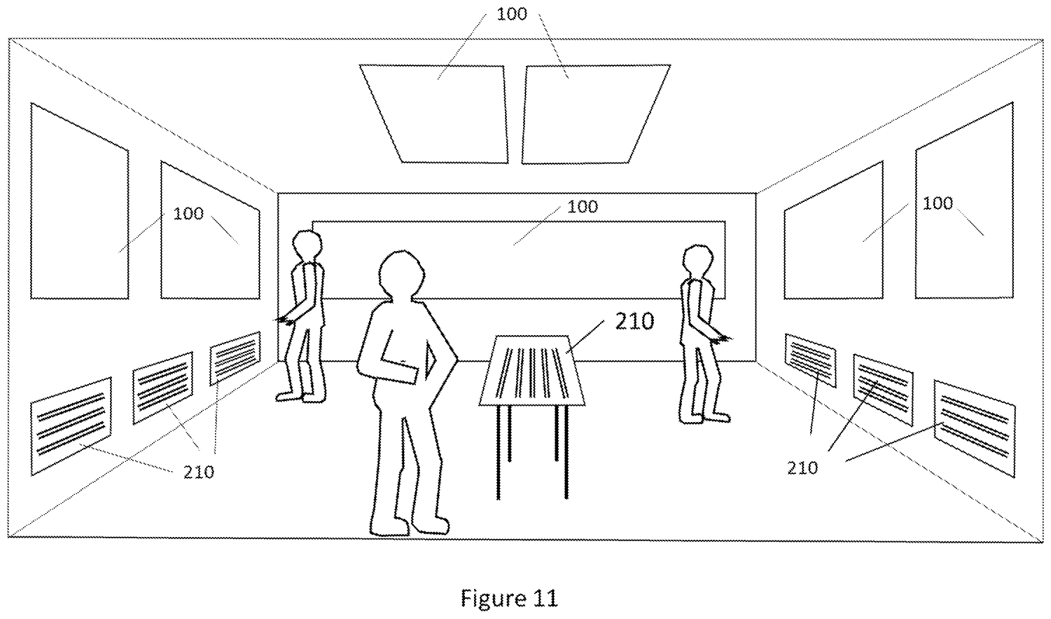

Like reference numbers are used throughout the figures to denote like components. Numerous components are illustrated schematically, it being understood that various details, connections and components of an apparent nature are not shown in order to emphasize features of the invention. Various features shown in the figures are not to drawn scale.

### DETAILED DESCRIPTION OF THE INVENTION

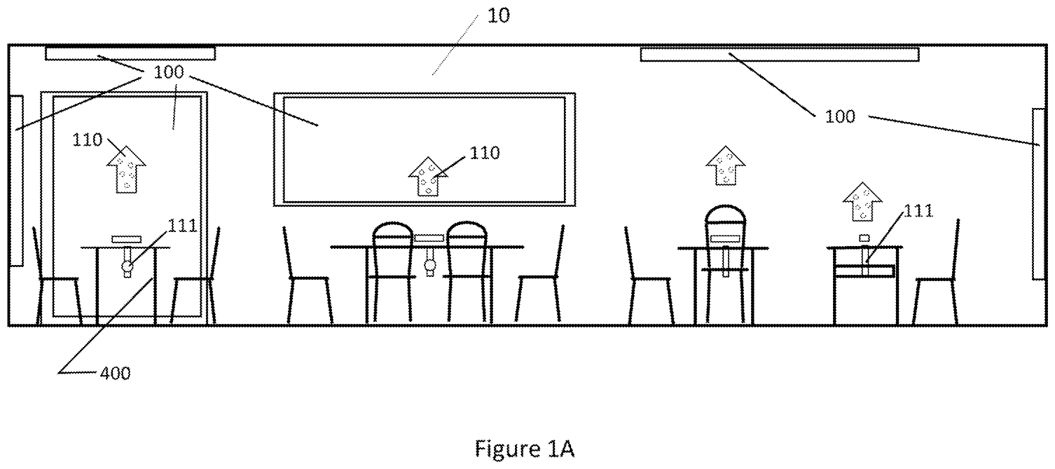

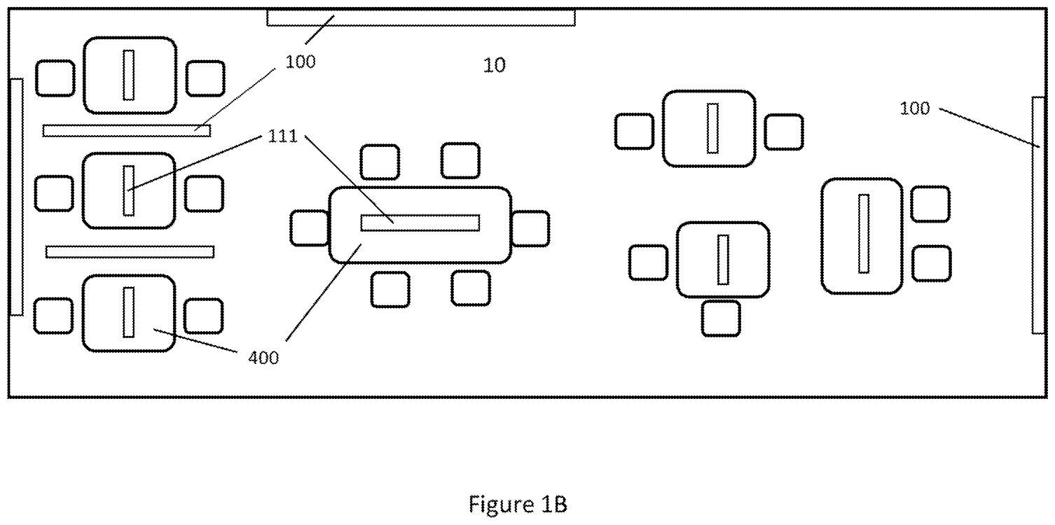

Example embodiments of the invention comprise modular capture systems 100 which can each be flexibly designed and scaled for optimal application in differing public and private space designs, e.g., indoor or outdoor spaces of varied size, and spaces in which there are differing arrangements of interacting persons. Referring to FIGS. 1 through 4, a system 10 are formed with one or more modular electrostatic capture units 100 each comprising a frame 101 which may function to provide support to an electromechanical assembly. Each modular unit in the system 10 may be of variable size, e.g., up to a 1.5 m width by up to a 3 m vertical height. The dimensions can be varied depending on aesthetic considerations and specific requirements of the space in which the system 10 is installed. For indoor applications, the size may depend on floor-to-ceiling height and furnished layout of, for example, a commercial dining area, a meeting hall, a theatre or auditorium or a classroom.

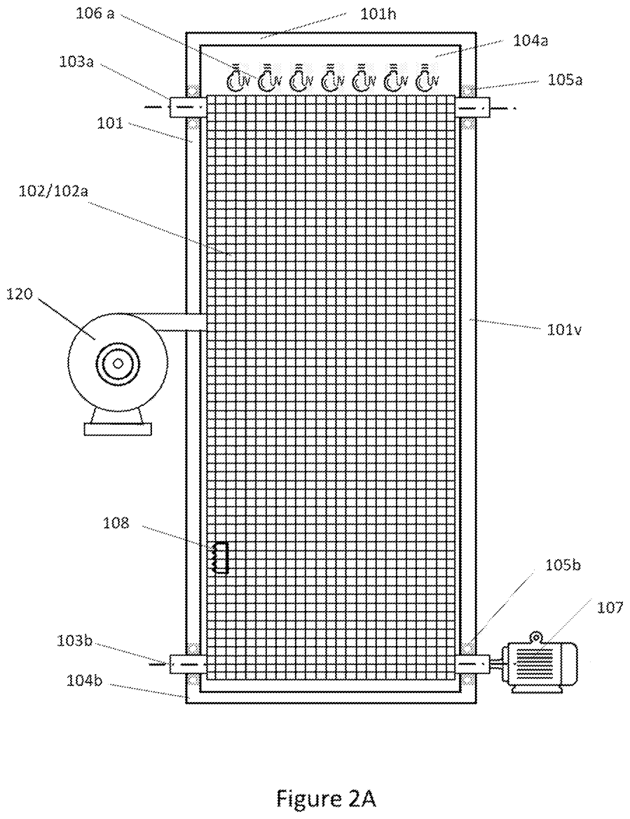

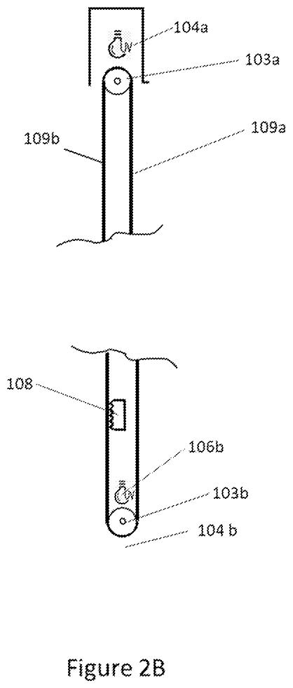

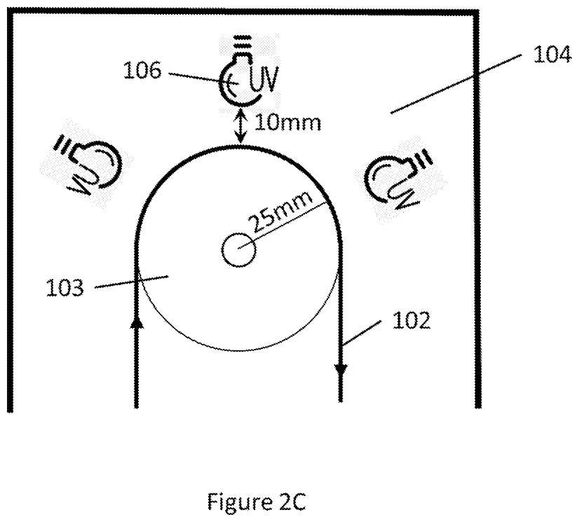

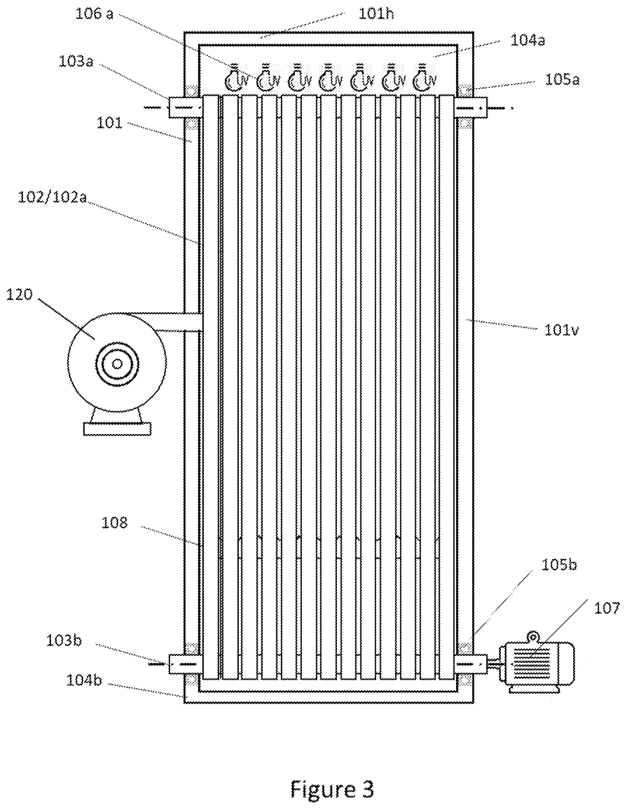

Referring to FIGS. 2 and 3, systems and methods according to the invention actively attract or direct movement of small particles, including pathogens, and then contain the particles over a relatively large defined surface area or volume. This may be effected with electrostatic forces or with directed air flows, to thwart transmission of pathogens between persons. The electrostatic forces may be generated within a region defined by the frame 101 or a housing, as described for illustrated embodiments, but the manner in which charge is accumulated is not so limited. The frame includes or adjoins one or plural housings or compartments, e.g., compartments 104a or 104b, also referred to as sanitizing chambers. The chambers 104 provide concealed cavities into which pathogens are transported and rendered noninfectious. The process reduces the probability of infection, especially by diverting and containing pathogens to limit further transport of the pathogens in aerosols and within enclosed environments.

The exemplary system 100 comprises frames 101 each having an exemplary modular size up to, for example, an effective area for capturing pathogens which may range at least between one and several square meters, with each unit flexibly designed and scaled for optimal application in differing public and private spaces, e.g., indoor or outdoor spaces of varied size, and for spaces in which there are differing arrangements and numbers of persons interacting in varied ways. In one example, the systems may extend up to a 3 m vertical height above a floor, with the dimensions depending in part on the floor to ceiling height and the furnished layout of, for example, a commercial dining area or a classroom.

The frame 101 may extend along a major plane defined by pairs of vertical and horizontal frame members 101v and 101h. Reference to vertical and horizontal members of a unit is made as though the unit is in a vertical orientation with respect to the ground plane, but it is to be understood that the units 100 may occupy a variety of orientations along or above the ground plane such as shown in FIGS. 1.

With reference to FIGS. 2a and 2b, each frame may contain a closed-loop rotatable sheet 102 which approximately spans the opening defined by the frame members. The illustrated sheet is connected for continuous rotation between upper and lower drums, 103a, 103b, journaled for rotation via upper and lower drum support bearings (105a, 105b). The drums may be driven by a variable speed electrical motor 107 coupled to the lower drum 103b. The unit 100 may be operated to provide continuous rotation of the sheet 102 as further described herein, but the unit 100 may also provide continual rotation of the sheet where sheet rotation may pause for predetermined durations of time to effect sequential application of sanitizing treatments to portions of the sheet 102 for specified continuous time exposures, before resuming movement of the sheet to expose another portion of the sheet for a predetermined amount of time.

The sheet 102 may comprise a highly insulating surface, or a conductive surface, charged to a suitable electrical potential. The portions of the surface passing along the aperture 102a are exposed to attract and capture small particles, e.g., aerosols having dimensions extending from the submicron range to a few microns. With the sheet 102 rotating, at any given moment of rotation varying surface segments of the sheet 102 are transported about upper and lower drum support bearings 105a, 105b. The moving surface segments of the sheet each provide varying forward-most surface portions 109a and varying rear-most surface portions 109b of the sheet. See the side view of the unit 100 shown in FIG. 2B.

With the sheet surface portions continuously moving, the exposed surface facing outward to receive and capture particles by electrostatic attraction, surface tension of the aerosol liquid or capillary effects, is continually or continuously changing. Similarly, different surface portions sequentially pass through compartments 104 which serve as sanitizing chambers. The varying forward-most surface portions 109a and varying rear-most surface portions 109b along the aperture 102a are also referred to as the front layer 109a and the rear layer 109b of the sheet. In one embodiment the sheet may be made of a wide-weave nylon fabric, having spaces on the order of 0.2 to 0.5 mm between fibers, which provide sufficient openings to allow airflow through both the front and rear layers 109a, 109b of the sheet 102.

A feature of the disclosed system is that the captured pathogens can be continually or continuously transported on and with the rotating fabric into a sanitizing chamber such as one of the upper or lower compartments 104 shown in the figures. Methods for sanitizing viral and bacterial pathogens include subjecting the pathogens to soft x-rays, plasmas, elevated temperatures, germicidal liquids and ultraviolet (UV) light in the range of 200 to 280 nm (referred to as UVC light). Of these sanitizing options, UVC light at 207 nm has similar antimicrobial properties as typical germicidal UVC light of 254 nm, but without inducing mammalian skin damage. The 207 nm wavelength appears to provide a safe, simple and cost-effective solution. Furthermore, since the rotating sheet 102 can enter and exit the sanitizing chamber through a very narrow slot, only a few mm wide or having a width only slightly greater than the thickness of the rotating sheet, the chamber 104 can be designed to permit only minimal amounts of UV light or no UV light to exit the chamber. The 207 nm UV radiation can, for example, be generated with commercially available UV LEDs placed in a range of 2 mm to 10 mm from the sheet 102, with the distance based on (i) the absorption/attenuation characteristics of UV light in air, (ii) the r⁻² inverse relationship between distance from the UVC light source and (iii) light intensity.

The necessary exposure to sanitize pathogens with UV light can be administered in, for example, multiples of ten second doses. The speed of the moving fabric passing through the sanitizing chamber 104 can be varied based on power output of the UV light sources and exposure time. Although it is advantageous to move the captured pathogens rapidly in and out of the sanitizing chamber, an exposure time of multiples of ten second doses may be required, depending on achievable delivery of power per unit area along the surface of the sheet 102, to assure provision of a sufficient dose to render the pathogen harmless.

Doses ranging from less than 0.5 Joule per square cm to more than one Joule per square cm are considered be reasonable and attainable, given the ability to incorporate multiple LED lamps in an array and cyclically expose the same area of the sheet 102 to multiple UVC doses. See FIG. 2C. Captured pathogens can be repeatedly cycled through one or multiple compartments 104a or 104b containing UV lights for sanitation. A slow-moving speed of the fabric in the range of one to five cm/second may be acceptable but, if necessary, slower speeds may be accommodated to reduce the number of cycles of sheet rotation required to render the pathogens non-infectious. The foregoing appears consistent with applying 300 to 600 millijoules of energy in 30 to 60 second exposure times to render pathogens non-infectious.

For an embodiment using UV radiation to disable pathogens, with the cyclic movement of the sheet 102, different portions of the sheet 102 sequentially move through the one or more compartments 104 containing UV light sources. With the varying sheet surface portions continuously moving, changing sheet portions continuously move through the one or more compartments containing UV light sources. Referring to FIG. 2C, an exemplary arrangement for the upper sanitizing chamber 104a includes a plurality of LED UV light sources 106 distributed to provide flux along the associated support drum 102a or 102b. Specifically, per FIG. 2C, flux is received from above the drum, along approximately a 180 degree arc. For a drum radius of 25 mm, the length of a portion of the sheet 102 being exposed to UVC light at any one time is about 78.5 mm. With a similar arrangement provided for the lower sanitizing chamber, a total of approximately 157 mm are simultaneously exposed. With a sheet rotational speed of 10 mm/sec, the exposure time for 157 mm of arc length of the sheet is 15.7 sec. Assuming that a one minute exposure time is sufficient to kill pathogens , about 4 complete rotations are needed. For a distance between the top of the roller 103a in the upper compartment 104a and the bottom of the roller 103b in the lower compartment 104b of one meter, the total time to accommodate this exposure is approximately 400 seconds.

Requisite dosage values for viruses in the same SARS virus family as SARS-CoV-2 is known to deliver 10-20 mJ/cm² using direct UVC light at a wavelength of 254 nm, rendering 99.9% of the pathogens non-infectious under controlled laboratory conditions. However, along the sheet 102, the virus may be partly concealed from receiving the full strength present in the UVC light directly incident on the sheet surface, thereby reducing the effective UVC dose. To compensate, dosages of 1,000-3,000 mJ/cm² may be applied to ensure 99.9% deactivation.

An alternate embodiment of the system 100, shown in FIGS. 3a and 3b, differs from the system 100 in that the closed-loop rotatable sheet 102 is replaced with multiple belt loops 102b of variable or uniform width, w, mounted under tension in mutually parallel orientations about the two drums 103a, 103b. To allow for an air flow through the set of belts, the belts 102b may be spaced apart by small gaps, e.g., 0.2 w. For example, the width, w, of the individual belts can be about 5 mm with gaps, g, of 1 mm between adjacent belts. In other embodiments, the belts may have perforations. The capture probability depends on particle size. That is, when the trajectory of particles is in the direction of a gap, the electrostatic force serves to bend the trajectory in order for the particle to hit the belt surface. This trajectory bending is increasingly difficult for larger physical momenta and therefore less likely to be effective for larger particles.

By way of example, each modular electrostatic capture unit 100 can have an effective surface area for capture of pathogens ranging from 0.5 square meters or less to several square meters to enhance the probability of capturing and passivating pathogens. Pathogen capture area refers to the area within the frame 101 of a single electrostatic capture unit 100 which can receive or capture pathogens. This may be approximately commensurate with the geometric area within the frame 101 and is referred to as the system aperture 102a. Generally, the inventive concepts can be deployed in a variety of venues of varying sizes with units that are modular or scalable implementations, this providing necessary flexibility for both optimal pathogen capture and economy.

In some embodiments the capturing probability can be enhanced by directing an airflow from the location of a person and toward the direction of the electrostatic capture unit 100 system. This airflow can be established with an air exhaust flow independent from operation of the electrostatic capture unit 100 or can be created with a pump attached to an electrostatic capture unit 100 to draw air toward the aperture 102a.

In the example embodiments of FIGS. 2-4, the system aperture 102a is a closed-loop rotatable sheet 102 which closely covers substantially, e.g., more than 95 percent of, the full area within the plane of the frame. In one embodiment the sheet can consist of a fabric comprising fine filaments, e.g., having diameters in the range of 0.01-0.1 mm of, for example, nylon, polyester or cotton. Nonwoven layers may also be suitable. During unit operation, tiny droplets coming into contact with the fabric are captured due to one or more effects such as the surface tension of the water droplet or capillary effects of the fine filaments. In some embodiments the capture of droplets can be enhanced by placing electrical charges on the fabric. A negative electrical charge can be established by spraying electrons onto the surface of the fabric. This can be done by applying a high voltage of several kV to sharp needles that point in the direction of the fabric, a technique described in. By creating high electrical fields about the small radii of the needle points, electrons can escape from the needles and be received on the insulating fabric, this resulting in a negative potential to ground. This approach is similar to the technique used in van de Graaff generators, where electrons are sprayed onto an insulating belt. In another embodiment the fabric can consist of a fine weave of thin copper wires, or a screen made of fine copper wires with a certain open area. In both embodiments, the copper fabric could be electrically charged by making an electrical connection to a high-voltage source establishing a potential of several kV with either positive or negative polarity relative to ground. Due to the small radius of a fine-diameter copper wire 0.1 to 0.3 mm, large electrical fields are generated in the vicinity of the wires which will attract the aerosol droplets. The well-established antimicrobial property of copper would help in the sanitization of pathogens captured by the fabric. In another embodiment, the fabric could be a material of the type used in fine mechanical filtering like HEPA filters.

For an insulative rotating sheet 102 or the belt loops 102b, the system 10 can be shown to be totally safe for humans due to the preclusion of significant current flow when the sheet or a belt loop comes into contact with a person's skin. For the conductive Cu sheet or fabric the connected power supply is highly current limited to a few micro amp. The sheet or the belts can be transparent or opaque and may contain pictures or artwork like a water fall to enhance visual aesthetics, or the modular units 100 may have color tones complementing the surrounding environment in order to blend in. To assure continuous movement, the drums 103a, 103b may be in mating engagement with the rotating sheet or belts analogous to operation of a toothed gear and chain. As an alternative or in addition to providing a mating engagement, tensioning of the sheet or belts may be sufficient and may add greater reliability to the rotational movement.

Charging of the insulating sheet or the belt loops can be effected in the same manner as the rubbing of a comb against a charge-collecting piece of fabric (triboelectric charging) or may be achieved with electrostatic generators, Marx-generators, or voltage multipliers which are well described in the literature. Such charging units 108 can spray electrons on the inside and outside surfaces of the sheet or the belt loops to produce a negative potential. In another embodiment, electrons can be extracted from the sheet or the belt loops causing a net positive potential. The electrical field generated in the vicinity of the single sheet 102 or the multiple belt loops 102b can provide sufficient attraction to cause any polarizable particles and any particles already carrying a small charge to stick to the rotating surface. This may include both pathogens and aerosol particles comprising the pathogens.

The particles which stick to the moving sheet or the belts are carried into an exemplary compartment 104a positioned above the top of the frame 101, e.g., along and above the upper horizontal frame member 101h. In one design, multiple UVC light sources 106 are enclosed within the concealed cavity of the compartment such that radiation is not transmitted exterior to the frame. The UV light is of sufficient flux density and photon energy to kill any pathogens attached to the sheet or the belt loops. UV-C light centered at a wavelength of 265 nm may provide the most effective action with reductions on either side of this spectral wavelength. Since the pathogens are attached by electrostatic forces to the rotating sheet or belt loops, it is not necessary to kill the pathogens on a single pass through the UV chamber. Complete elimination of pathogens can be affected with multiple passes of rotation through the upper compartment 104a.

Instead of or in addition to providing a chamber of UV radiation along the top or bottom of the frame 101, other processes lethal to pathogens can be employed after the pathogens are positioned on the sheet or the belt loops. For example, a chemical bath having lethal effects on the pathogens can be placed in a lower compartment 104b positioned along the lower horizontal frame member 101h, e.g., in a reservoir. The bath may contain, for example, a bleach solution, alcohol or other germicidal chemical.

The UV light sources 106 shown in the upper compartment 104a in FIGS. 2A and 3A can primarily affect particles on the outer surface of the sheet or the belt loops. However, the effectiveness of the electrostatic capture unit 100 can be augmented with an air ionizer located between the front layer 109a and back layer 109b of the rotatable sheet or belt loops. An air ionizer (not shown in the figures) adds electrons to otherwise neutral or slightly charged particles entering the volume between the two layers. To capture the resulting negatively charged particles, the sheet or the belt loops are positively charged. Addition of an ionizer enhances the probability of capturing particles passing through the rotating sheet or belts. Since these particles may then become attached to the inside surface of the sheet or the belt loops, they may be most effectively be rendered noninfectious by coming into contact with a chemical bath in the lower compartment 104b. With the ionizer located between the two layers (109a and 109b) the problem with conventional air ionizers is avoided, i.e., ionized particles may otherwise stick to any surface region encountered on the sheet 102. If ionized pathogens stick to surfaces like the walls of a room, they may still infect people on contact. In embodiments having the ionizer located on the inside of the rotating sheet or the belt loops 102, no human contact with the captured pathogen occurs.

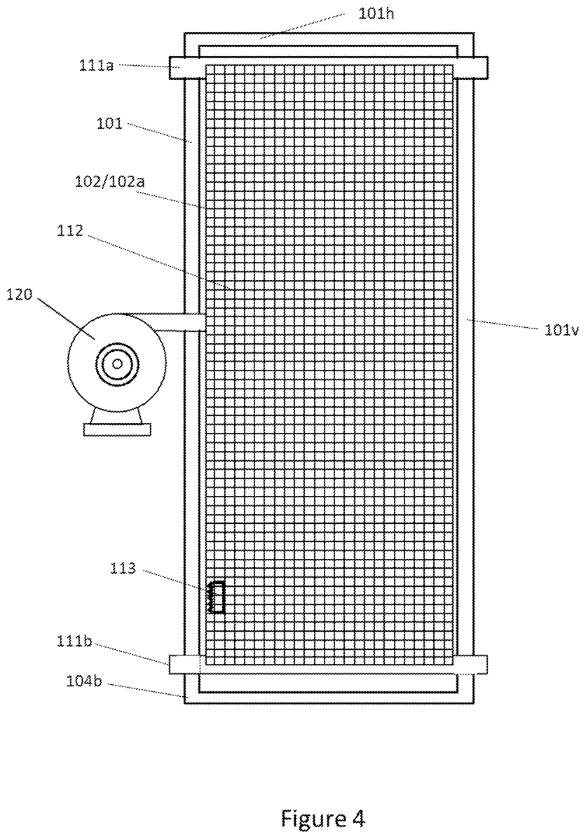

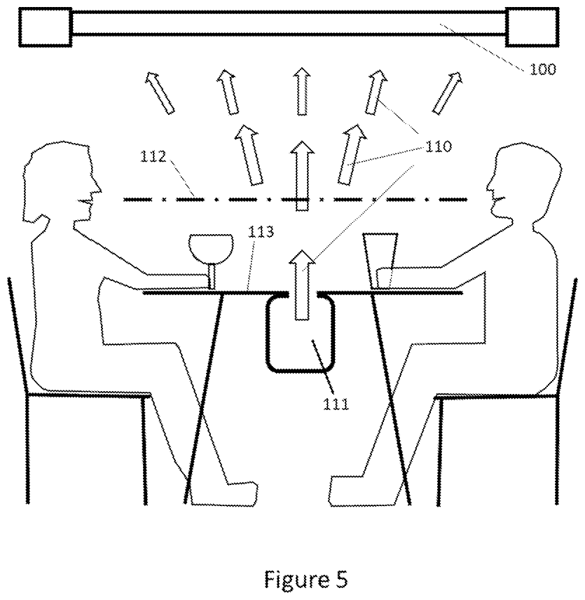

For optimum performance of the electrostatic capture system, i.e., for intercepting and removing pathogens from a given closed environment, a well-controlled air flow advantageously minimizes or prevents accumulation and distribution of pathogen-containing aerosols, this avoiding situations where air exhaled from one person can be directly inhaled by another person. In environments where people gather for extended periods of time it is helpful to provide localized and directed air drafts that prevent transmission of exhaled air from person to person. FIGS. 4 and 5 illustrate example embodiments where two people sit opposite each other at a table, or more than two people sit around tables such as when having dinner in a restaurant.

To significantly reduce pathogen contamination and prevent a flow path of pathogens from person to person in this situation a directed local air flow is established. By directed air flow it is meant that an active means, such as a blower fan or air intake in combination with custom configured ducting creates a dominant flow of air that limits or prevents transmission of airborne pathogens from one person directly to another person. With two people sitting opposite each other at a table, and without controlled airflows as shown in FIGS. 4 and 5, pathogen-containing aerosols can pass without impediment directly from person to person. To modify the direct paths of aerosols between persons across or adjacent one another at the table, a predominant upward directed airflow 110 is established, e.g., perpendicular to an exemplary horizontal line 112 connecting the faces of the two people shown in FIGS. 4 and 5. For this embodiment a constant airflow in the upward direction exiting from the table surface is established.

By way of example, the airflow may be established by a blower system connected to channel air to an air duct 111 located under the table surface 113. One purpose of the continuous airflow is to intercept exhaled air moving between people sitting at the table. The flow rate of the channeled air may therefore be adjusted to effect this purpose. Assuming that the air surrounding the table is not already contaminated with pathogens, this air can be used as an input to the blower system. In some embodiments a HEPA filter or a UV-C light source can be placed in line with the airflow exiting from the table. If a new facility is being built, an airduct system can be installed that supplies multiple tables in the room with sterilized air as an extra precaution.

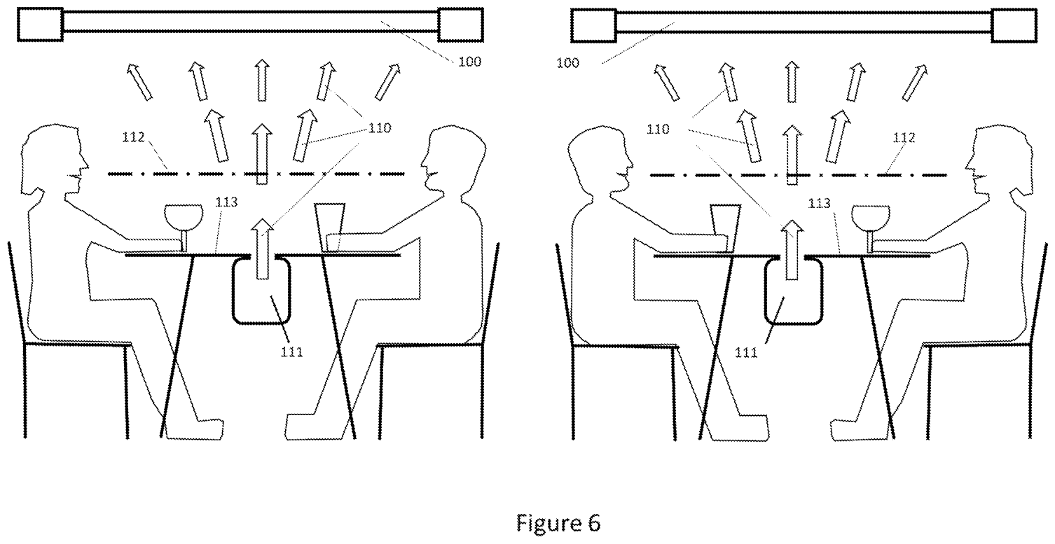

For the described configurations, a modular electrostatic capture unit 100 is positioned above the heads of the people seated at a table. In some embodiments the capture unit 100 can be equipped with a pump to further enhance the airflow toward it. As further described herein, other embodiments may replace the sheet 100 of the electrostatic capture unit with copper or copper alloy in the form of a perforated sheet, a flexible screen mesh or a layer of woven copper-containing fabric, to provide well-established antimicrobial effects of materials comprising copper. When the copper material is provided in the form of a flexible sheet, a screen or a fabric, the electrostatic capture unit 100 can include a rotating assembly and one or more compartments that provide a germicidal process such as afore described irradiation with UV lights. Referring to FIG. 5, in still another embodiment the vents ______ ##______ establish an airflow in a horizontal direction 110, perpendicular to horizontal line 112, with an electrostatic capture system 100 and/or a copper sheet placed along a side of the table to receive and sterilize the airflow. The sterilized air from the capture system is simply released on the side of the capture system since the air can be completely sterilized. FIG. 6 illustrates application of the invention to two adjacent restaurant booths with the airflow 110 moving in an exemplary vertical direction, and a separating wall 113, positioned between the two booths, formed with a modular frame 101.

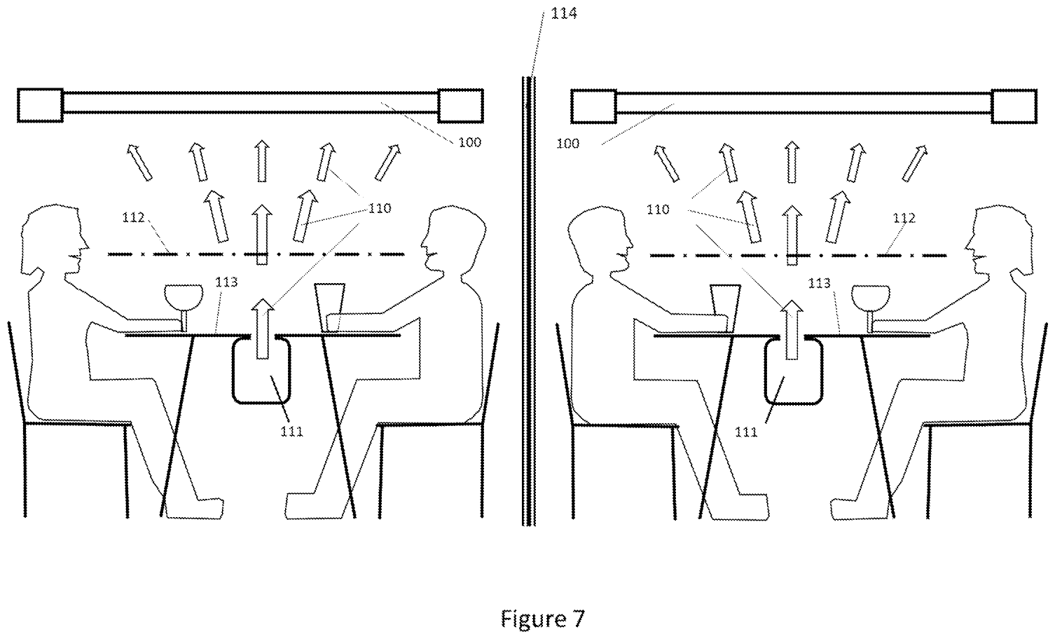

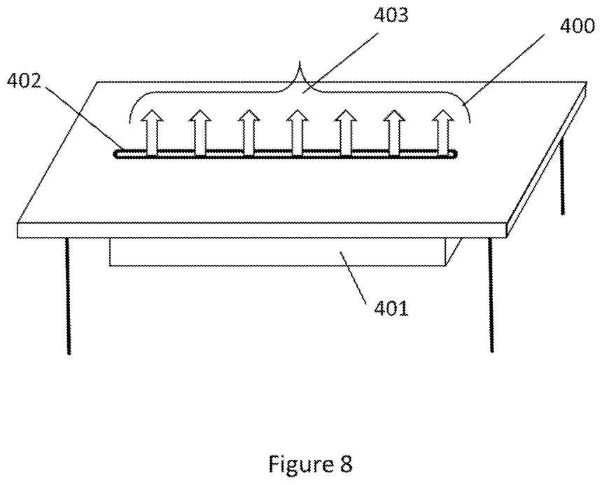

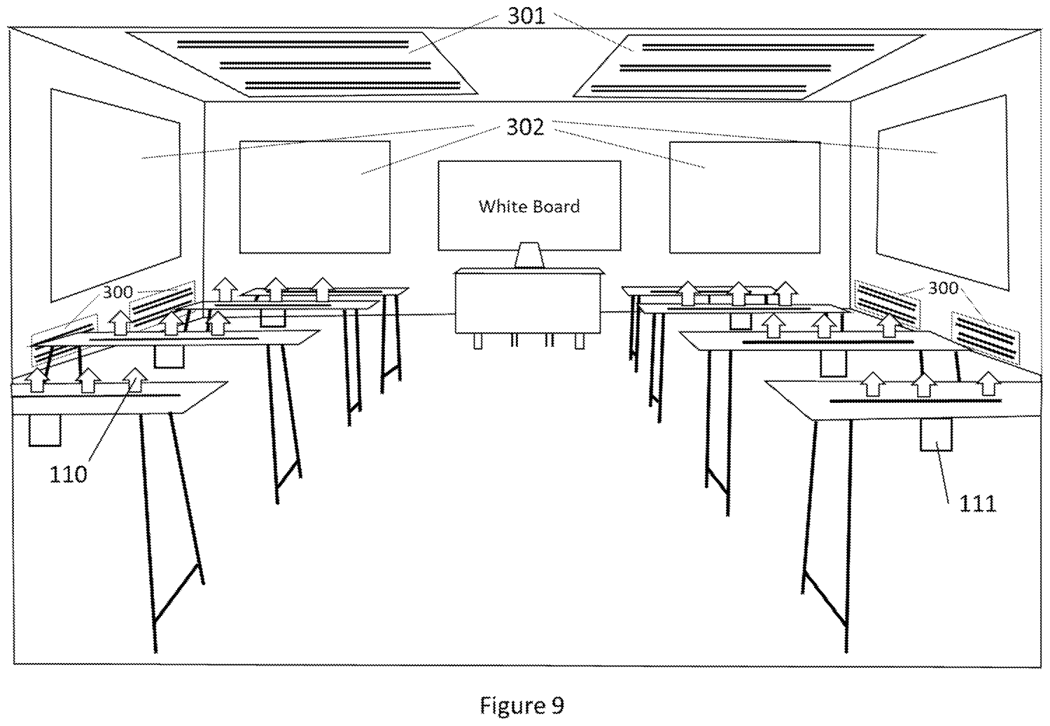

Design principles which integrate a localized airflow within a table, where people spend extended periods of time, are directly transferable to desks placed in open office spaces, classroom desks, high tables in bars and arrangements in which people stand around tables, bars and the like. The desks and tables for these applications are schematically shown in FIGS. 7, 8 and 9. These desks and tables can be equipped with a blower system 401, located under the tabletop or, for new construction, air outlets can be placed in the floor to establish the required airflow out of the tabletop. In general, for facilities not yet established, the optimum airflow system can best be integrated during the design phase incorporating, for example, ceiling and floor duct systems which recirculate air through duct segments illuminated with UVC light. In all these embodiments a capture system is best placed overhead, drawing an upward directed airflow. In many cases it can be sufficient and most economical to use a copper or copper alloy sheet instead of an electrostatic capture unit 100, again using the well-established antimicrobial properties of copper. The described embodiments of the invention are independent of existing air conditioning installations and can be integrated in existing locations.

For some embodiments, a pump 120 is integrated in the electrostatic capture system, taking air in from one or both sides of the rotating sheet (109a and 109b) and releasing the sterilized air on top of the system, or porting the sterilized air toward the floor for recirculation. This configuration may serve as a separating wall between cubicles in offices or between booths in fast food restaurants.

For open environments, like museums and gymnasiums, or where people pass one another along aisles, airflow and capture systems comprising the units 100 can be located on opposing walls, establishing a laminar airflow between a system which emits an air flow and a system which captures the emitted air flow. In an alternate embodiment, small pumps, placed in distributed stands throughout a large room, generate upward-directed air streams and electrostatic capture systems and/or copper sheets, placed along the ceiling and above the stands. These can be an effective solution to avoid accumulation and contamination of a large area. In such embodiments, additional air sterilization can be integrated in an existing air conditioning system with UV lights.

All described embodiments are only exemplary and can be modified and optimized for given environments.

The disclosed systems for avoiding contamination and accumulation of airborne pathogens in enclosed environments are mechanically simple, reliable, and inexpensive. The systems can be introduced in existing facilities like schools, offices, restaurants, and any locations where people gather for extended periods of time. By both continuously sterilizing and controlling the flow of the air, the potential for infections, which is proportional to the product of exposure time and pathogen concentration, is significantly reduced. Many facilities can safely be used, perhaps even with full occupancy when the probability of pathogen transfer from person to person is significantly reduced.

The claimed invention is:

1. A method for reducing the infections from a pathogen by influencing transport with an electric field effect based on a charge associated with the pathogen or responsive to a electric charge associated with a particle comprising the pathogen, where the effect may be based on net charge or based on charge polarization between or among atoms or molecules associated with the pathogen or the particle, the method comprising:
positioning a material in a location where the pathogen may be transmitted from a person and through a volume of air about the person;
providing the material with a net electric charge sufficient to create a measurable electric field extending from the material; and
to the extent the pathogen becomes positioned along a portion of the material, applying a treatment about the material portion to disable the infectious nature of the pathogen.

2. The method of claim 1 where the treatment is provided to the material portion by transporting the material portion to a region which isolates the effects of the treatment from one or more persons present about the volume of air.

3. The method of claim 1 where the pathogen is part of an aerosol particle.

4. A treatment system for rendering a pathogen non-infectious within an open area, comprising:
a sheet positionable in the open area and through which an air flow can pass;
a ventilation device positionable to impart an air flow between a person present in the open area and the sheet to direct movement of air emitted by the person toward the sheet, wherein a mechanism or characteristic associated with the sheet is of sufficient strength to cause attachment to the sheet of a particle in the air flow which includes the pathogen; and
a radiant source positioned to provide a sufficient dose of radiant flux directed to the sheet such that, when the pathogen is attached to the sheet, the radiation dose renders the pathogen non-infectious.

5. The system of claim 4 where the sheet is rotatable to sequentially move portions of the sheet past the radiant source, the sheet comprising any of a porous fabric, a woven fabric, and a screen, mesh or fabric comprising copper.

6. The system of claim 4 where the mechanism or characteristic provides a force due to the presence of an electric field, this resulting in sufficient attraction to cause an airborne polarizable particle, or an airborne particle carrying a net charge, to stick to a surface of the sheet.

7. The system of claim 4 where the radiant source is taken from the group consisting of a UV-C light source, a microwave generator, an infrared source, and resistive wire positioned in or along the layer of material.

8. A treatment process for rendering a pathogen non-infectious, comprising:
in a predefined area, actively redirecting an air flow emitted by a person toward a sheet exposed to an air current in the predefined area and through which a portion of the air flow and air current can pass;
when a pathogen is present in the air flow it is redirected by the air current toward the sheet, attaching the pathogen to the sheet; and
while the pathogen is on the sheet, applying a treatment process directly to the pathogen to render the pathogen non-infectious.

9. The process of claim 8 where the attaching results from a force due to presence of an electric field, surface tension or capillary effects.

10. A system to reduce contact with an infectious airborne pathogen transmitted from a person, comprising:
a pair of spaced apart rollers, each journaled for rotation;
a layer of material positioned to move along a plane extending about both rollers for movement between the rollers; and
a radiant source positioned to direct radiant flux toward a first of the rollers.

11. The system of claim 10 where the radiant source is taken from the group consisting of a UV-C light source, a microwave generator, an infrared source, and resistive conductor positioned in or along the layer of material.

12. The system of claim 10 where the material is a porous sheet taken from the group consisting of natural and synthetic fabrics, insulators, conductors, rubber, polyesters and plastics.

13. The system of claim 10 where the material is a sheet comprising copper in the form of a screen, a woven fabric or a perforated sheet.

14. The system of claim 10 further including a shield positioned adjacent the first roller to limit emanation of the radiant flux in multiple directions away from the first roller and thereby block a portion of the radiant flux along a path toward a person positioned near the system.

15. The system of claim 14 where:
the shield is formed about the first roller to provide a cavity into which the layer of material extends;
the rollers are journaled for rotation; and
the layer is connected for rotation about the rollers as the rollers rotate, this enabling sequential movement of portions of the layer cyclically into and out of the cavity to receive radiant flux.

16. The system of claim 14 configured to provide continuous motorized rotation of the layer.

17. The system of claim 14 configured to provide continual motorized rotation of the layer that sequentially positions a first portion of the layer in a stationary position to receive the radiant flux for a predetermined time period before positioning a second portion of the layer in a stationary position.
